(19) United States
(12) Patent Application Publication
(10) Pub. No.: US 2020/0253907 A1
(43) Pub. Date: Aug. 13, 2020

(54) Compositions and Methods for Treating Neurodegenerative Diseases

(71) Applicant: The Regents of the University of California, Oakland, CA (US)

(72) Inventors: Jing Huang, Los Angeles, CA (US); Min Chai, Los Angeles, CA (US); Brett Eugene Lomenick, Los Angeles, CA (US); Meisheng Jiang, Thousand Oaks, CA (US); Randall M. Chin, Van Nuys, CA (US)

(21) Appl. No.: 16/639,676

(22) PCT Filed: Aug. 20, 2018
(86) PCT No.: PCT/IB2018/056272

§ 371 Date: Feb. 17, 2020

Related U.S. Application Data
(60) Provisional application No. 62/548,324, filed on Aug. 21, 2017.

(51) Int. Cl. A61K 31/198; A61K 31/194; A61K 31/19; A61P 25/28

(52) U.S. Cl. CPC A61K 31/198; A61P 25/28; A61K 31/19; A61K 31/194

(57) ABSTRACT

Disclosed herein are methods and compounds for treating neurodegenerative diseases and disorders with one or more neurodegenerative therapeutics and/or amino acid derivatives or analogs.

### BACKGROUND OF THE INVENTION

### 1. Field of the Invention

The present invention relates to compositions and methods for treating neurodegenerative diseases and disorders.

### 2. Description of the Related Art

Alzheimer's disease is a chronic neurodegenerative disease with an initial slow rate of disease progression. There are about 48 million cases of Alzheimer's disease worldwide. There is no cure for Alzheimer's disease, and in some cases, treatments focused on temporarily symptom improvement.

### SUMMARY OF THE INVENTION

In some embodiments, the present invention provides compositions and methods of treating a neurodegenerative disease or disorder. In some embodiments, the methods comprise treating a neurodegenerative disease or disorder in a subject in need thereof, which comprises administering to the subject a pharmaceutical composition comprising a therapeutically effective amount of one or more compounds of Formula I:

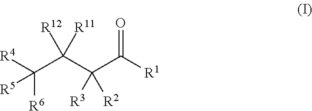

wherein R¹ is hydrogen, halogen, —CHO, —OR⁷, —NR⁸R⁹, —COOR⁷, —CONR⁸R⁹, —SR¹⁰, substituted or unsubstituted alkyl, or substituted or unsubstituted heteroalkyl; R² and R³ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; or R² and R³, together with the atom to which they are bound, form an oxo; R¹¹ and R¹² are each independently hydrogen, halogen, —CN, —CHO, —OR¹³, —NR¹⁴R¹⁵, —COOR¹³, —CONR¹⁴R¹⁵, —NO₂, —SR¹⁶, substituted or unsubstituted alkyl, or substituted or unsubstituted heteroalkyl; R⁴, R⁵, and R⁶ are each independently hydrogen, halogen, —CN, —CHO, —OR⁷, —NR⁸R⁹, —COOR⁷, —CONR₈R⁹, —NO₂, —SR¹⁰, substituted or unsubstituted alkyl, or substituted or unsubstituted heteroalkyl; R⁷, R⁸, R⁹, and R¹⁰ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R¹³, R¹⁴, R¹⁵, and R¹⁶ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or a salt thereof. In some embodiments, R¹ is hydrogen, —CHO, or —OR⁷. In some embodiments, R¹ is —OR⁷, wherein R⁷ is hydrogen, substituted or unsubstituted alkyl, or substituted or unsubstituted aryl. In some embodiments, R¹ is hydrogen. In some embodiments, R¹ is —OR⁷, wherein R⁷ is C₁₋₂₀ substituted or unsubstituted alkyl. In some embodiments, R² is hydrogen, halogen, —CN, —CHO, —OR⁷, or —NR⁸R⁹, wherein R⁷, R⁸ and R⁹ are each independently hydrogen or substituted or unsubstituted alkyl. In some embodiments, R² is hydrogen, —OR⁷, or —NR⁸R⁹, wherein R⁷, R⁸ and R⁹ are each independently hydrogen or substituted or unsubstituted alkyl. In some embodiments, R³ is hydrogen, halogen, —CN, —CHO, —OR⁷, or —NR⁸R⁹, wherein R⁷, R⁸ and R⁹ are each independently hydrogen or substituted or unsubstituted alkyl. In some embodiments, R³ is hydrogen, —OR⁷, or —NR⁸R⁹, wherein R⁷, R⁸ and R⁹ are each independently hydrogen or substituted or unsubstituted alkyl. In some embodiments, R² and R³, together with the atom to which they are bound, form an oxo. In some embodiments, R¹¹ is hydrogen, halogen, —CN, —CHO, —OR¹³, —NR¹⁴R¹⁵, or substituted or unsubstituted alkyl, wherein R¹³, R¹⁴ and R¹⁵ are each independently hydrogen or substituted or unsubstituted alkyl. In some embodiments, R¹¹ is hydrogen, —OR¹³, or —NR¹⁴R¹⁵, wherein R¹³, R¹⁴ and R¹⁵ are each independently hydrogen or substituted or unsubstituted alkyl. In some embodiments, R¹² is hydrogen, halogen, —CN, —CHO, —OR¹³, —NR¹⁴R¹⁵, or substituted or unsubstituted alkyl, wherein R¹³, R¹⁴ and R¹⁵ are each independently hydrogen or substituted or unsubstituted alkyl. In some embodiments, R¹² is hydrogen, —OR¹³, or —NR¹⁴R¹⁵, wherein R¹³, R¹⁴ and R¹⁵ are each independently hydrogen or substituted or unsubstituted alkyl. In some embodiments, R⁴, R⁵, and R⁶ are each independently hydrogen, —CHO, —OR⁷, —NR⁸R⁹, —COOR⁷, or —CONR⁸R⁹, wherein R⁷, R⁸, and R⁹ are each independently hydrogen or C₁₋₂₀ substituted or unsubstituted alkyl. In some embodiments, R⁴ is —COOR⁷ or —CONR⁸R⁹, wherein R⁷, R⁸, and R⁹ are each independently hydrogen or C₁₋₂₀ substituted or unsubstituted alkyl. In some embodiments, R⁵ and R⁶ are each independently hydrogen, —OR⁷, or —CONR⁸R⁹, wherein R⁷, R⁸, and R⁹ are each independently hydrogen or C₁₋₂₀ substituted or unsubstituted alkyl. In some embodiments, the neurodegenerative disease or disorder is a neurodegenerative disease of the central nervous system. In some embodiments, the neurodegenerative disease or disorder is Alzheimer's disease, Parkinson's disease, or Huntington disease. In some embodiments, the neurodegenerative disease or disorder is Alzheimer's disease. In some embodiments, the compound of Formula I is alpha-ketoglutarate (α-KG). In some embodiments, the compound of Formula I is 2-HB. In some embodiments, the compound of Formula I is α-ketobutyrate (α-KB). In some embodiments, the compound of Formula I is α-ketoisocaproate (KIC). In some embodiments, the compound of Formula I is α-ketoisovalerate (KIV). In some embodiments, the concentration of α-KG is about 1 μM to about 16 mM. In some embodiments, the concentration of α-KG is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, or about 16 mM. In some embodiments, the concentration of 2-HB is about 1 μM to about 16 mM. In some embodiments, the concentration of 2-HB is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, or about 16 mM. In some embodiments, the concentration of α-KB is about 1 μM to about 16 mM. In some embodiments, the concentration of α-KB is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, or about 16 mM. In some embodiments, the concentration of KIC is about 1 μM to about 32 mM. In some embodiments, the concentration of KIC is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, about 15 mM, about 16 mM, about 20 mM, about 24 mM, about 25 mM, about 28 mM, about 30 mM, or about 32 mM. In some embodiments, the concentration of KIV is about 1 μM to about 32 mM. In some embodiments, the concentration of KIV is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, about 15 mM, about 16 mM, about 20 mM, about 24 mM, about 25 mM, about 28 mM, about 30 mM, or about 32 mM. In some embodiments, the method further comprises administering to the subject a pharmaceutical composition comprising alanine. In some embodiments, the concentration of alanine is about 1 μM to about 16 mM. In some embodiments, KIC is administered simultaneously with alanine. In some embodiments, KIC is administered sequentially with alanine. In some embodiments, KIV is administered simultaneously with alanine. In some embodiments, KIV is administered sequentially with alanine. In some embodiments, the compound of Formula I is formulated for oral or parenteral administration. In some embodiments, the therapeutically effective amount is administered as a single dose. In some embodiments, the therapeutically effective amount is administered in at least two doses, at least three doses, at least four doses, at least five doses, or more. In some embodiments, the therapeutically effective amount is administered daily. In some embodiments, the therapeutically effective amount is administered every other day. In some embodiments, the subject is a human.

In some embodiments, the present invention provides a method of treating a neurodegenerative disease or disorder in a subject in need thereof which comprises administering to the subject a first pharmaceutical composition comprising α-ketoisocaproate (KIC) and/or α-ketoisovalerate (KIV), and an excipient. In some embodiments, the method further comprises administering to the subject a second pharmaceutical composition comprising alanine. In some embodiments, the concentration of KIC is about 1 μM to about 32 mM. In some embodiments, the concentration of KIC is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, about 15 mM, about 16 mM, about 20 mM, about 24 mM, about 25 mM, about 28 mM, about 30 mM, or about 32 mM. In some embodiments, the concentration of KIV is about 1 μM to about 32 mM. In some embodiments, the concentration of KIV is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, about 15 mM, about 16 mM, about 20 mM, about 24 mM, about 25 mM, about 28 mM, about 30 mM, or about 32 mM. In some embodiments, the concentration of alanine is about 1 μM to about 16 mM. In some embodiments, KIC is administered simultaneously with alanine. In some embodiments, KIC is administered sequentially with alanine. In some embodiments, KIV is administered simultaneously with alanine. In some embodiments, KIV is administered sequentially with alanine. In some embodiments, the neurodegenerative disease or disorder is a neurodegenerative disease of the central nervous system. In some embodiments, the neurodegenerative disease or disorder is Alzheimer's disease, Parkinson's disease, or Huntington disease. In some embodiments, the neurodegenerative disease or disorder is Alzheimer's disease. In some embodiments, the first pharmaceutical composition is formulated for oral or parenteral administration. In some embodiments, the second pharmaceutical composition is formulated for oral or parenteral administration. In some embodiments, the first pharmaceutical composition and/or the second pharmaceutical composition is administered as a therapeutically effective amount. In some embodiments, the therapeutically effective amount is administered as a single dose. In some embodiments, the therapeutically effective amount is administered in at least two doses, at least three doses, at least four doses, at least five doses, or more. In some embodiments, the therapeutically effective amount is administered daily. In some embodiments, the therapeutically effective amount is administered every other day. In some embodiments, the subject is a human.

The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

Color versions of these drawings may be obtained from the United States Patent & Trademark Office (USPTO) in U.S. Application No. 62/548,324 and can also be obtained from “WorldWideWeb.canadylortz.com/wp-content/uploads/2018/08/034044.174WO1-figures.pdf”, wherein WorldWideWeb=“www”.

This invention is further understood by reference to the drawings wherein:

FIG. 1, FIG. 2, FIG. 3, FIG. 4, FIG. 5, and FIG. 6 illustrate a decrease in Aβ toxicity and delayed paralysis in GMC101 C. elegans strain induced by small molecules. FIG. 1 shows anti-aging compound, α-ketoglutarate (α-KG) can extend the health span of GMC101 C. elegans strain at 2 mM. From left to right, the first line is vehicle. FIG. 2 shows α-ketobutyrate (α-KB) can delay Aβ caused paralysis, e.g., at a concentration of about 2 mM. At Day 2, from top to bottom, the lines are 2 mM α-KB, 8 mM α-KB, 4 mM α-KB, and vehicle. FIG. 3 shows an exemplary α-KB analog, 2-hydroxybutyrate (2-HB) can also delay GMC101 paralysis at 4 mM. A delay in paralysis is not observed with 3-hydroxybutyrate (3-HB). At Day 2, from top to bottom, the lines are 4 mM 2-HB, vehicle, and 4 mM 3-HB. FIG. 4 shows that alanine can reduce paralysis caused by Aβ. At Day 3, the first set of overlapping lines from top to bottom are 2 mM, 4 mM, 8 mM, and the second set of overlapping lines from top to bottom are 1 mM and vehicle. FIG. 5 shows that the metabolite of leucine, α-ketoisocaproate (KIC) reduces paralysis caused by Aβ. At Day 4, from top to bottom, the lines are 4 mM, 2 mM, 8 mM, 16 mM, vehicle, and 32 mM. FIG. 6 shows that pyruvate reduces Aβ toxicity and delays paralysis. The metabolite of valine, α-ketoisovalerate (KIV) can extend GMC101 health span. At Day 3, from top to bottom, the lines are 8 mM pyruvate, 8 mM KIV, and vehicle.

FIG. 7, FIG. 8, FIG. 9, FIG. 10, and FIG. 11 illustrate that 16 mM KIC can synergize with alanine to decrease Aβ toxicity and delay paralysis in GMC101 C. elegans strain. FIG. 7 shows that 16 mM KIC can delay paralysis when treated together with alanine, while 16 mM KIC is not observed to exhibit beneficial effects in GMC101. At Day 4, the lines from top to bottom, are 16 mM KIC+4 mM Alanine, 16 mM KIC+2 mM Alanine, 16 mM KIC+8 mM Alanine, 16 mM KIC+1 mM Alanine, and the last two overlapping lines from top to bottom are 16 mM KIC, and vehicle. FIG. 8 shows that 1 mM alanine does not synergize with 16 mM KIC in GMC101. On Day 3, the top line is 16 mM KIC+1 mM Alanine. FIG. 9, FIG. 10, and FIG. 11 show 2 mM, 4 mM, or 8 mM alanine can extend health span of GMC101 and synergize with 16 mM KIC to reduce paralysis caused by Aβ. In FIG. 9, at Day 4, from top to bottom, the lines are 16 mM KIC+2 mM Alanine, 2 mM Alanine, and the bottom overlapping lines are 16 mM KIC and vehicle. In FIG. 10, at Day 4, from top to bottom, the lines are 16 mM KIC+4 mM Alanine, 4 mM Alanine, and the bottom overlapping lines are 16 mM KIC and vehicle. In FIG. 11, at Day 4, from top to bottom, the lines are 16 mM KIC+8 mM Alanine, 8 mM Alanine, and the bottom overlapping lines are 16 mM KIC and vehicle.

FIG. 12, FIG. 13, FIG. 14, and FIG. 15 show 4 mM alanine can synergize with KIC only at high concentration to decrease Aβ toxicity and delay paralysis in GMC101 C. elegans strain. FIG. 12 shows 4 mM alanine can delay paralysis when treated together with 16 mM KIC, while lower concentrations of KIC is not observed to exert a beneficial effect in combination with alanine. At Day 4, the top line is 4 mM Alanine+16 mM KIC, the middle lines from top to bottom are 4 mM Alanine+8 mM KIC, 4 mM Alanine+2 mM KIC, and 4 mM Alanine, and the bottom line is vehicle. FIG. 13 and FIG. 14 show 2 mM or 8 mM KIC does not synergize with 4 mM alanine in GMC101. In FIG. 13, at Day 2, from top to bottom, the lines are 4 mM Alanine+2 mM KIC, 4 mM Alanine, 2 mM KIC, and vehicle. In FIG. 14, at Day 3, from top to bottom, the lines are 4 mM Alanine+8 mM KIC, 4 mM Alanine, vehicle, and 8 mM KIC. FIG. 15 shows 16 mM KIC does not exert a beneficial effect in GMC101 but is observed to synergize with 4 mM alanine to reduce paralysis caused by Aβ. At Day 3, from top to bottom, the lines are 4 mM Alanine+16 mM KIC, 4 mM Alanine, vehicle, and 16 mM KIC.

FIG. 16, FIG. 17, and FIG. 18 show that 200 μM octyl α-KG can protect against amyloid-beta induced neuronal cell death. FIG. 16 shows 200 μM octyl α-KG reduces the percentage of differentiated PC-12 neurons that died after treatment with Aβ peptide. FIG. 17 shows that 200 μM octyl α-KG protects isolated primary mouse hippocampal neurons from cell death induced by Aβ peptide treatment. FIG. 18 shows that 200 μM octyl α-KG reduces the percentage of isolated primary rat hippocampal neurons that died after treatment with Aβ peptide.

### DETAILED DESCRIPTION OF THE INVENTION

Neurodegenerative diseases and disorders comprise a group of conditions characterized by progressive neuronal cell death. Neurodegenerative diseases and disorders are further classified into neurodegenerative disease of the central nervous system (or systemic atrophies primarily affecting the central nervous system), dementia, motor neuron disease, Tay-Sachs disease, and transmissible spongiform encephalopathies.

As disclosed herein, the present invention is directed to methods and compositions for the treatment of neurodegenerative diseases and disorders. As used herein, a “neurodegenerative disease or disorder” (neurodegenerative disease or disorder) refers to a condition characterized by progressive neuronal cell death and neurodegenerative diseases and disorders broadly include neurodegenerative diseases of the central nervous system, dementia, motor neuron disease, Tay-Sachs disease, and transmissible spongiform encephalopathies. Examples of neurodegenerative diseases and disorders include Alzheimer's disease, Parkinson's disease, amyotrophic lateral sclerosis (ALS), spinal muscular atrophies, Tay-Sachs disease, Creutzfeldt-Jakob disease (CJD) and its variant (vCJD), Gerstmann-Strussler-Scheinker syndrome, fatal familial insomnia, and kuru. In some embodiments, the neurodegenerative disease or disorder is a neurodegenerative disease of the central nervous system. In some embodiments, the neurodegenerative disease or disorder is Alzheimer's disease, Parkinson's disease, or Huntington disease. In some embodiments, the neurodegenerative disease or disorder is Alzheimer's disease.

In some embodiments, the present invention is directed to methods and compositions for the treatment of a neurodegenerative disease or disorder. In some embodiments, the present invention provides a method of treating a neurodegenerative disease or disorder in a subject in need thereof, which comprises administering to the subject a therapeutically effective amount of one or more neurodegenerative therapeutics such as α-ketoisocaproate (KIC) and/or α-ketoisovalerate (KIV). In some embodiments, the one or more neurodegenerative therapeutics are administered in the form of a pharmaceutical composition.

In some embodiments, the present invention is directed to methods and compositions for the treatment of Alzheimer's disease. In some embodiments, the present invention provides a method of treating Alzheimer's disease in a subject in need thereof, which comprises administering to the subject a therapeutically effective amount of one or more neurodegenerative therapeutics such as α-ketoisocaproate (KIC) and/or α-ketoisovalerate (KIV). In some embodiments, the one or more neurodegenerative therapeutics are administered in the form of a pharmaceutical composition.

In some embodiments, the present invention is directed to methods and compositions for the treatment of Parkinson's disease. In some embodiments, the present invention provides a method of treating Parkinson's disease in a subject in need thereof, which comprises administering to the subject a therapeutically effective amount of one or more neurodegenerative therapeutics such as α-ketoisocaproate (KIC) and/or α-ketoisovalerate (KIV). In some embodiments, the one or more neurodegenerative therapeutics are administered in the form of a pharmaceutical composition.

In some embodiments, the present invention is directed to methods and compositions for the treatment of Huntington disease. In some embodiments, the present invention provides a method of treating Huntington disease in a subject in need thereof, which comprises administering to the subject a therapeutically effective amount of one or more neurodegenerative therapeutics such as α-ketoisocaproate (KIC) and/or α-ketoisovalerate (KIV). In some embodiments, the one or more neurodegenerative therapeutics are administered in the form of a pharmaceutical composition.

In some embodiments, the treatment methods slow down or inhibit the progression of the neurodegenerative disease or disorder and/or alleviate or reduce one or more symptoms of the neurodegenerative disease or disorder as compared to a control, e.g., the average rate of disease progression of untreated subjects, the severity of symptoms prior to treatment, etc.

### Neurodegenerative Therapeutics

As used herein, a “neurodegenerative therapeutic” refers to a compound having the following Formula I (which includes α-KG compounds, 2-HG compounds, 2-HB compounds, α-ketoisocaproate (KIC), and α-ketoisovalerate (KIV)):

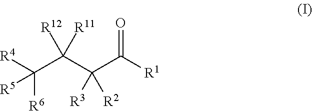

wherein

R¹ is hydrogen, halogen, —CHO, —OR⁷, —NR⁸R⁹, —COOR⁷, —CONR⁸R⁹, —SR¹⁰, substituted or unsubstituted alkyl, or substituted or unsubstituted heteroalkyl;

R² and R³ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl; or R² and R³, together with the atom to which they are bound, form an oxo;

R¹¹ and R¹² are each independently hydrogen, halogen, —CN, —CHO, —OR¹³, —NR¹⁴R¹⁵, —COOR¹³, —CONR¹⁴R¹⁵, —NO₂, —SR¹⁶, substituted or unsubstituted alkyl, or substituted or unsubstituted heteroalkyl;

R⁴, R⁵, and R⁶ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl;

R⁷, R⁸, R⁹, and R¹⁰ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

R¹³, R¹⁴, R¹⁵, and R¹⁶ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,

and pharmaceutically acceptable solvates, pharmaceutically acceptable salts, pharmaceutically acceptable prodrugs, and pharmaceutically active metabolites thereof.

In some embodiments, R¹ is hydrogen, —CHO, or —OR⁷. In some embodiments, R¹ is —OR⁷, wherein R⁷ is hydrogen, substituted or unsubstituted alkyl, or substituted or unsubstituted aryl. In some embodiments, R¹ is hydrogen. In some embodiments, R¹ is —OH. In some embodiments, R¹ is —OR⁷, wherein R⁷ is C₁₋₂₀ substituted or unsubstituted alkyl. In some embodiments, R¹ is —OR⁷, wherein R⁷ is C₁₋₁₀ unsubstituted alkyl. In some embodiments, R¹ is —OR⁷, wherein R⁷ is methyl, ethyl, or propyl. In some embodiments, R¹ is hydrogen, —CHO, or —OR⁷. In some embodiments, R¹ is —OR⁷, wherein R⁷ is hydrogen, substituted or unsubstituted alkyl, or substituted or unsubstituted aryl.

In some embodiments, R² is hydrogen, halogen, —CN, —CHO, —OR⁷, or —NR⁸R⁹, wherein R⁷, R⁸, and R⁹ are each independently hydrogen or substituted or unsubstituted alkyl. In some embodiments, R² is hydrogen, —OR⁷, or —NR⁸R⁹, wherein R⁷, R⁸, and R⁹ are each independently hydrogen or substituted or unsubstituted alkyl. In some embodiments, R² is hydrogen. In some embodiments, R² is —NR⁸R⁹, wherein R⁸ and R⁹ are each independently hydrogen or substituted or unsubstituted alkyl. In some embodiments, R² is —NR⁸R⁹, wherein R⁸ and R⁹ are each hydrogen.

In some embodiments, R³ is hydrogen, halogen, —CN, —CHO, —OR⁷, or —NR⁸R⁹, wherein R⁷, R⁸, and R⁹ are each independently hydrogen or substituted or unsubstituted alkyl. In some embodiments, R³ is hydrogen, —OR⁷, or —NR⁸R⁹, wherein R⁷, R⁸, and R⁹ are each independently hydrogen or substituted or unsubstituted alkyl. In some embodiments, R³ is hydrogen. In some embodiments, R³ is —NR⁸R⁹, wherein R⁸ and R⁹ are each independently hydrogen or substituted or unsubstituted alkyl. In some embodiments, R³ is —NR⁸R⁹, wherein R⁸ and R⁹ are each hydrogen. In some embodiments, R² and R³, together with the atom to which they are bound, form an oxo.

In some embodiments, R¹¹ is hydrogen, halogen, —CN, —CHO, —OR¹³, —NR¹⁴R¹⁵, or substituted or unsubstituted alkyl, wherein R¹³, R¹⁴, and R¹⁵ are each independently hydrogen or substituted or unsubstituted alkyl. In some embodiments, R¹¹ is hydrogen, —OR¹³, or —NR¹⁴R¹⁵, wherein R¹³, R¹⁴, and R¹⁵ are each independently hydrogen or substituted or unsubstituted alkyl. In some embodiments, R¹¹ is hydrogen. In some embodiments, R¹¹ is —OR¹³, wherein R¹³ is hydrogen or substituted or unsubstituted alkyl. In some embodiments, R¹¹ is —NR¹⁴R¹⁵, wherein R¹⁴ and R¹⁵ are each independently hydrogen or substituted or unsubstituted alkyl. In some embodiments, R¹¹ is —NR¹⁴R¹⁵, wherein R¹⁴ and R¹⁵ are each hydrogen.

In some embodiments, R¹² is hydrogen, halogen, —CN, —CHO, —OR¹³, —NR¹⁴R¹⁵, or substituted or unsubstituted alkyl, wherein R¹³, R¹⁴, and R¹⁵ are each independently hydrogen or substituted or unsubstituted alkyl. In some embodiments, R¹² is hydrogen, —OR¹³, or —NR¹⁴R¹⁵, wherein R¹³, R¹⁴, and R¹⁵ are each independently hydrogen or substituted or unsubstituted alkyl. In some embodiments, R¹² is hydrogen. In some embodiments, R¹² is —OR¹³, wherein R¹³ is hydrogen or substituted or unsubstituted alkyl. In some embodiments, R¹² is —NR¹⁴R¹⁵, wherein R¹⁴, and R¹⁵ are each independently hydrogen or substituted or unsubstituted alkyl. In some embodiments, R¹¹ is —NR¹⁴R¹⁵, wherein R¹⁴ and R¹⁵ are each hydrogen.

In some embodiments, R⁴, R⁵, and R⁶ are each independently hydrogen, —CHO, —OR⁷, —NR⁸R⁹, —COOR⁷, or —CONR⁸R⁹, wherein R⁷, R⁸, and R⁹ are each independently hydrogen or C₁₋₂₀ substituted or unsubstituted alkyl. In some embodiments, R⁴ is —COOR⁷ or —CONR⁸R⁹, wherein R⁷, R⁸, and R⁹ are each independently hydrogen or C₁₋₂₀ substituted or unsubstituted alkyl. In some embodiments, R⁴ is hydrogen. In some embodiments, R⁵, and R⁶ are each independently hydrogen, —OR⁷, or —CONR⁸R⁹, wherein R⁷, R⁸, and R⁹ are each independently hydrogen or C₁₋₂₀ substituted or unsubstituted alkyl. In some embodiments, R⁵ is hydrogen. In some embodiments, R⁶ is hydrogen.

As used herein, an “α-KG compound” refers to α-ketoglutarate (α-KG), derivatives of α-ketoglutarate (e.g., the derivatives set forth in MacKenzie, et al. (2007) Mol Cell Biol 27(9):3282-3289)), analogues of α-ketoglutarate (e.g., phosphonate analogues (e.g., those recited in Bunik, et al. (2005) Biochemistry 44(31): 10552-61), esters of α-ketoglutarate (e.g., dimethyl α-ketoglutarate and octyl α-ketoglutarate), and various species specific analogues, e.g., human α-ketoglutarate, porcine α-ketoglutarate, murine α-ketoglutarate, bovine α-ketoglutarate, and the like. In some embodiments, the α-KG compound is α-ketoglutaric acid. In some embodiments, the α-KG compound is dimethyl α-ketoglutarate. In some embodiments, the α-KG compound is octyl α-ketoglutarate. As used herein, “α-ketoglutarate” and “α-ketoglutaric acid” are used interchangeably. As used herein, the abbreviation “KG” may be used to refer to the term “ketoglutarate”, e.g., α-ketoglutarate is abbreviated as α-KG.

As used herein, a “2-HG compound” refers to 2-hydroxyglutaric acid, 2-hydroxypentanedioate, and compounds having 2-hydroxypentanedioate as part of its backbone structure and includes 1-alkyl-(S)-2-hydroxypentanedioate, 1-alkyl-(R)-2-hydroxypentanedioate, 1-alkenyl-(S)-2-hydroxypentanedioate, 1-alkenyl-(R)-2-hydroxypentanedioate, 5-alkyl-(S)-2-hydroxypentanedioate, 5-alkyl-(R)-2-hydroxypentanedioate, 5-alkenyl-(S)-2-hydroxypentanedioate, and 5-alkenyl-(R)-2-hydroxypentanedioate, wherein alkyl is a straight or branched C₁-C₁₀ alkyl and alkenyl is a straight or branched C₁-C₁₀ alkenyl. In some embodiments, the 2-HG compound is 1-octyl-(S)-2-hydroxypentanedioate, 1-octyl-(R)-2-hydroxypentanedioate, 5-octyl-(S)-2-hydroxypentanedioate, or 5-octyl-(R)-2-hydroxypentanedioate. In some embodiments, the 2-HG compound is disodium (S)-2-hydroxyglutarate or (S)-2-hydroxyglutaric acid (S-2HG). In some embodiments, the 2-HG compound is L-α-hydroxyglutaric acid disodium salt. As used herein, the abbreviation “HG” may be used to refer to the term “hydroxypentanedioate”, e.g., 2-hydroxypentanedioate is abbreviated as 2-HG. As used herein, “2-hydroxyglutarate” and “2-hydroxyglutaric acid” are used interchangeably. In some embodiments, a 2-HG compound is S-2HG.

As used herein, a “2-HB compound” includes 2-hydroxybutyrate and derivatives thereof.

A “pharmaceutically acceptable solvate” refers to a solvate form of a specified compound that retains the biological effectiveness of the specified compound. Examples of solvates include compounds of the invention in combination with water, isopropanol, ethanol, methanol, dimethyl sulfoxide, ethyl acetate, acetic acid, ethanolamine, or acetone. Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of compounds of neurodegenerative therapeutics are within the scope of the invention. It will also be appreciated by those skilled in organic chemistry that many organic compounds can exist in more than one crystalline form. For example, crystalline form may vary from solvate to solvate. Thus, all crystalline forms of the neurodegenerative therapeutics or the pharmaceutically acceptable solvates thereof are within the scope of the present invention.

The term “pharmaceutically acceptable salt” refers to a salt form that is pharmacologically acceptable and substantially non-toxic to the subject being treated with the compound of the invention. Pharmaceutically acceptable salts include acid-addition salts or base-addition salts formed from suitable non-toxic organic or inorganic acids or inorganic bases known in the art. Exemplary acid-addition salts include those derived from inorganic acids such as hydrochloric acid, hydrobromic acid, hydroiodic acid, sulfuric acid, sulfamic acid, phosphoric acid, and nitric acid, and those derived from organic acids such as p-toluenesulfonic acid, methanesulfonic acid, ethane-disulfonic acid, isethionic acid, oxalic acid, p-bromophenylsulfonic acid, carbonic acid, succinic acid, citric acid, benzoic acid, 2-acetoxybenzoic acid, acetic acid, phenylacetic acid, propionic acid, glycolic acid, stearic acid, lactic acid, malic acid, tartaric acid, ascorbic acid, maleic acid, hydroxymaleic acid, glutamic acid, salicylic acid, sulfanilic acid, and fumaric acid. Exemplary base-addition salts include those derived from ammonium hydroxides (e.g., a quaternary ammonium hydroxide such as tetramethylammonium hydroxide), those derived from inorganic bases such as alkali or alkaline earth-metal (e.g., sodium, potassium, lithium, calcium, or magnesium) hydroxides, and those derived from non-toxic organic bases such as basic amino acids.

A “pharmaceutically acceptable prodrug” is a compound that may be converted under physiological conditions or by solvolysis to the specified compound or to a pharmaceutically acceptable salt of such compound. A “pharmaceutically active metabolite” refers to a pharmacologically active product produced through metabolism in the body of a specified compound or salt thereof. Prodrugs and active metabolites of a compound may be identified using techniques known in the art. See, e.g., Bertolini, G. et al., (1997) J. Med. Chem. 40:2011-2016; Shan, D. et al., J. Pharm. Sci., 86(7):765-767; Bagshawe K., (1995) Drug Dev. Res. 34:220-230; Bodor, N., (1984) Advances in Drug Res. 13:224-331; Bundgaard, H., Design of Prodrugs (Elsevier Press, 1985) and Larsen, I. K., Design and Application of Prodrugs, Drug Design and Development (Krogsgaard-Larsen et al., eds., Harwood Academic Publishers, 1991).

### Treatments

In some embodiments, the present invention provides a method of treating a neurodegenerative disease or disorder in a subject in need thereof which comprises administering to the subject one or more neurodegenerative therapeutics. In some embodiments, a therapeutically effective amount of the one or more neurodegenerative therapeutics is administered to the subject. In some embodiments, the one or more neurodegenerative therapeutics are administered in the form of a pharmaceutical composition. Preferably the subject is mammalian, more preferably, the subject is human. As used herein, a subject “in need of” treatment is one who has a family history of neurodegenerative diseases and disorders, has a biomarker for a neurodegenerative disease or disorder, exhibits one or more symptoms of a neurodegenerative disease or disorder, and/or has been clinically diagnosed as having a neurodegenerative disease or disorder. In some embodiments, the neurodegenerative disease or disorder is Alzheimer's disease. In some embodiments, the neurodegenerative disease or disorder is Parkinson's disease. In some embodiments, the neurodegenerative disease or disorder is Huntington disease.

In some embodiments, the present invention provides a method of treating neurodegenerative disease or disorder in a subject in need thereof which comprises administering to the subject a pharmaceutical composition which comprises at least one neurodegenerative therapeutic, which is a compound of Formula I:

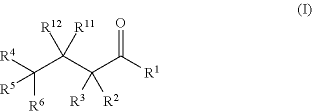

wherein

R¹ is hydrogen, —OR⁷, —NR⁸R⁹, —COOR⁷, —CONR⁸R⁹, —SR¹⁰, substituted or unsubstituted alkyl, or substituted or unsubstituted heteroalkyl;

R² and R³ are each independently hydrogen, —OR⁷, —NR⁸R⁹, —COOR⁷, —CONR⁸R⁹, or substituted or unsubstituted alkyl;

or R² and R³, together with the atom to which they are bound, form an oxo;

R¹¹ and R¹² are each independently hydrogen, —OR¹³, —NR¹⁴R¹⁵, —COOR¹³, —CONR¹⁴R¹⁵, or substituted or unsubstituted alkyl;

R⁴, R⁵, and R⁶ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl;

R⁷, R⁸, R⁹, and R¹⁰ are each independently hydrogen, substituted or unsubstituted alkyl, substituted or unsubstituted heteroalkyl, substituted or unsubstituted cycloalkyl, or substituted or unsubstituted heterocycloalkyl; and

R¹³, R¹⁴, R¹⁵, and R¹⁶ are each independently hydrogen, substituted or unsubstituted alkyl, substituted or unsubstituted heteroalkyl, substituted or unsubstituted cycloalkyl, or substituted or unsubstituted heterocycloalkyl; and pharmaceutically acceptable solvates, salts, prodrugs, and metabolites thereof; and a pharmaceutically acceptable carrier.

In some embodiments, the present invention provides a method of treating neurodegenerative disease or disorder in a subject in need thereof which comprises administering to the subject a pharmaceutical composition comprising at least one neurodegenerative therapeutic, which is a compound of Formula I:

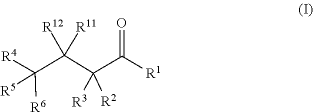

wherein

R¹ is hydrogen, —OR⁷, —COOR⁷, or substituted or unsubstituted alkyl;

R² and R³ are each independently hydrogen, —OR⁷, —NR⁸R⁹, or substituted or unsubstituted alkyl;

or R² and R³, together with the atom to which they are bound, form an oxo;

R¹¹ and R¹² are each independently hydrogen, —OR¹³, —NR¹⁴R¹⁵, or substituted or unsubstituted alkyl;

R⁴, R⁵, and R⁶ are each independently hydrogen, —OR⁷, —NR⁸R⁹, —COOR⁷, —CONR⁸R⁹, substituted or unsubstituted alkyl, or substituted or unsubstituted heteroalkyl;

R⁷, R⁸, R⁹, and R¹⁰ are each independently hydrogen, or substituted or unsubstituted alkyl; and

R¹³, R¹⁴, R¹⁵, and R¹⁶ are each independently hydrogen, substituted or unsubstituted alkyl, or substituted or unsubstituted heteroalkyl; and pharmaceutically acceptable solvates, salts, prodrugs, and metabolites thereof; and a pharmaceutically acceptable carrier.

In some embodiments, the present invention provides a method of treating neurodegenerative disease or disorder in a subject in need thereof which comprises administering to the subject a pharmaceutical composition comprising at least one neurodegenerative therapeutic, which is a compound of Formula I:

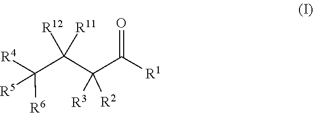

wherein

R¹ is hydrogen or —OR⁷;

R² and R³ are each independently hydrogen, —OR⁷, or —NR⁸R⁹;

or R² and R³, together with the atom to which they are bound, form an oxo;

R¹¹ and R¹² are each independently hydrogen or substituted or unsubstituted alkyl;

R⁴, R⁵, and R⁶ are each independently hydrogen or substituted or unsubstituted alkyl, or substituted or unsubstituted heteroalkyl;

R⁷, R⁸, R⁹, and R¹⁰ are each independently hydrogen, or substituted or unsubstituted alkyl; and

R¹³, R¹⁴, R¹⁵, and R¹⁶ are each independently hydrogen, or substituted or unsubstituted alkyl; and pharmaceutically acceptable solvates, salts, prodrugs, and metabolites thereof; and a pharmaceutically acceptable carrier.

In some embodiments, the present invention provides a method of treating neurodegenerative disease or disorder in a subject in need thereof which comprises administering to the subject a pharmaceutical composition comprising at least one neurodegenerative therapeutic, which is a compound represented by the structure:

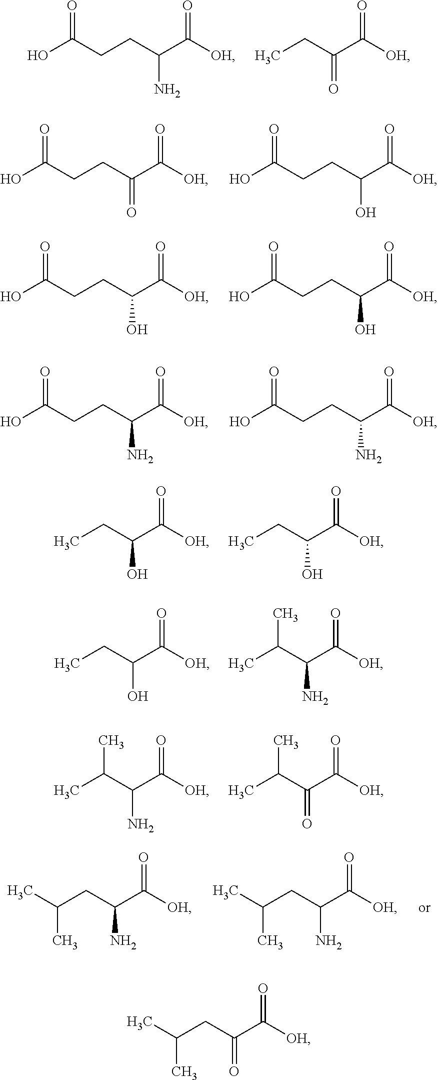

and pharmaceutically acceptable solvates, salts, prodrugs, and metabolites thereof; and a pharmaceutically acceptable carrier.

In some embodiments, the present invention provides a method of treating neurodegenerative disease or disorder in a subject in need thereof which comprises administering to the subject a pharmaceutical composition comprising at least one neurodegenerative therapeutic, which is a compound represented by the structure:

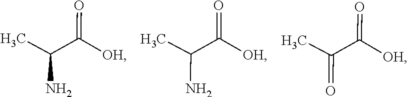

and pharmaceutically acceptable solvates, salts, prodrugs, and metabolites thereof; and a pharmaceutically acceptable carrier.

In some embodiments, the present invention provides a method of treating neurodegenerative disease or disorder in a subject in need thereof which comprises administering to the subject a pharmaceutical composition comprising at least one neurodegenerative therapeutic selected from: alpha-ketoglutarate (α-KG), glutamate, alpha-hydroglutarate (α-HG), R-2-hydroglutarate, S-2-hydroglutarate, alpha-ketobutyrate (α-KB), 2-hydrobutyrate (2-HG), R-2-hydrobutyrate, S-2-hydrobutyrate, valine, leucine, alpha-ketoisocaproate (KIC), alpha-ketoisovalerate (KIV), alanine, pyruvate, and pharmaceutically acceptable solvates, salts, prodrugs, and metabolites thereof; and a pharmaceutically acceptable carrier.

### Combination Therapy

In some embodiments, the present invention provides a method of treating neurodegenerative disease or disorder in a subject in need thereof which comprises administering to the subject a therapeutically effective amount one or more neurodegenerative therapeutics in combination with at least one amino acid or a pharmaceutically acceptable salt of any one thereof. In some embodiments, the one or more neurodegenerative therapeutics and the at least one amino acid or salts thereof are administered as separate pharmaceutical compositions. In some embodiments, the one or more neurodegenerative therapeutics and the at least one amino acid or salts thereof are formulated together and administered as a single pharmaceutical composition.

In some embodiments, the neurodegenerative therapeutic is KIC. In some embodiments, the concentration of KIC is about 1 μM to about 32 mM. In some embodiments, the concentration of KIC is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, about 15 mM, about 16 mM, about 20 mM, about 24 mM, about 25 mM, about 28 mM, about 30 mM, or about 32 mM. In some embodiments, the concentration of KIC is about 1 μM. In some embodiments, the concentration of KIC is about 10 μM. In some embodiments, the concentration of KIC is about 100 μM. In some embodiments, the concentration of KIC is about 500 μM. In some embodiments, the concentration of KIC is about 1 mM. In some embodiments, the concentration of KIC is about 1.5 mM. In some embodiments, the concentration of KIC is about 2 mM. In some embodiments, the concentration of KIC is about 2.5 mM. In some embodiments, the concentration of KIC is about 3 mM. In some embodiments, the concentration of KIC is about 4 mM. In some embodiments, the concentration of KIC is about 5 mM. In some embodiments, the concentration of KIC is about 8 mM. In some embodiments, the concentration of KIC is about 10 mM. In some embodiments, the concentration of KIC is about 15 mM. In some embodiments, the concentration of KIC is about 16 mM. In some embodiments, the concentration of KIC is about 20 mM. In some embodiments, the concentration of KIC is about 24 mM. In some embodiments, the concentration of KIC is about 25 mM. In some embodiments, the concentration of KIC is about 28 mM. In some embodiments, the concentration of KIC is about 30 mM. In some embodiments, the concentration of KIC is about 32 mM.

In some embodiments, the neurodegenerative therapeutic is KIV. In some embodiments, the concentration of KIV is about 1 μM to about 32 mM. In some embodiments, the concentration of KIV is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, about 15 mM, about 16 mM, about 20 mM, about 24 mM, about 25 mM, about 28 mM, about 30 mM, or about 32 mM. In some embodiments, the concentration of KIV is about 1 μM. In some embodiments, the concentration of KIV is about 10 μM. In some embodiments, the concentration of KIV is about 100 μM. In some embodiments, the concentration of KIV is about 500 μM. In some embodiments, the concentration of KIV is about 1 mM. In some embodiments, the concentration of KIV is about 1.5 mM. In some embodiments, the concentration of KIV is about 2 mM. In some embodiments, the concentration of KIV is about 2.5 mM. In some embodiments, the concentration of KIV is about 3 mM. In some embodiments, the concentration of KIV is about 4 mM. In some embodiments, the concentration of KIV is about 5 mM. In some embodiments, the concentration of KIV is about 8 mM. In some embodiments, the concentration of KIV is about 10 mM. In some embodiments, the concentration of KIV is about 15 mM. In some embodiments, the concentration of KIV is about 16 mM. In some embodiments, the concentration of KIV is about 20 mM. In some embodiments, the concentration of KIV is about 24 mM. In some embodiments, the concentration of KIV is about 25 mM. In some embodiments, the concentration of KIV is about 28 mM. In some embodiments, the concentration of KIV is about 30 mM. In some embodiments, the concentration of KIV is about 32 mM.

In some embodiments, the at least one amino acid is selected from alanine, asparagine, aspartic acid, arginine, cysteine, glutamine, glycine, glutamic acid, histidine, isoleucine, lysine, leucine, phenylalanine, methionine, serine, proline, tryptophan, threonine, tyrosine, valine, and pharmaceutically acceptable salts thereof. In some embodiments, the amino acid is alanine. In some embodiments, the concentration of alanine is about 1 μM to about 16 mM. In some embodiments, the concentration of alanine is about 1 μM. In some embodiments, the concentration of alanine is about 10 μM. In some embodiments, the concentration of alanine is about 100 μM. In some embodiments, the concentration of alanine is about 500 μM. In some embodiments, the concentration of alanine is about 1 mM. In some embodiments, the concentration of alanine is about 2 mM. In some embodiments, the concentration of alanine is about 4 mM. In some embodiments, the concentration of alanine is about 5 mM. In some embodiments, the concentration of alanine is about 8 mM. In some embodiments, the concentration of alanine is about 10 mM. In some embodiments, the concentration of alanine is about 12 mM. In some embodiments, the concentration of alanine is about 15 mM. In some embodiments, the concentration of alanine is about 16 mM.

In some embodiments, KIC is administered with a pharmaceutical composition comprising alanine. In some embodiments, KIC is administered simultaneously with alanine. In some embodiments, KIC is administered sequentially with alanine. In some embodiments, KIC is administered prior to administering alanine. In some embodiments, KIC is administered after administering alanine.

In some embodiments, KIV is administered with a pharmaceutical composition comprising alanine. In some embodiments, KIV is administered simultaneously with alanine. In some embodiments, KIV is administered sequentially with alanine. In some embodiments, KIV is administered prior to administering alanine. In some embodiments, KIV is administered after administering alanine.

In some embodiments, the amount of KIC and/or KIV, and the amount of the at least one amino acid, e.g., alanine, or salt thereof administered to a subject or provided in a pharmaceutical composition are synergistically effective amounts. As used herein, a “synergistically effective amount” of a first compound in relation to a given amount of a second compound is an amount that results in an activity that is greater than the sum of the individual activity of the first compound and the individual activity of the second compound. Synergistically effective amounts can be determined using the methods described herein. For example, the lower and upper endpoints of the range of amounts of a given amino acid or salt thereof, e.g., alanine, that provide synergistic therapeutic results when administered in combination a given amount of a given neurodegenerative therapeutic, and vice versa, can determined by the experiments described in Example 1 below.

### Additional Therapeutic Agent

The one or more compounds disclosed herein to be administered to a subject may be administered with an additional agent. In some embodiments, the additional agent comprises an agent for the treatment of Alzheimer's disease. In some embodiments, the additional agent comprises an agent for the treatment of Parkinson's disease. In some embodiments, the additional agent comprises an agent for the treatment of Huntington disease. In some embodiments, exemplary additional agents include cholinesterase inhibitors such as donepezil, rivastigmine, or galantamine; amantadine; apomorphine; benztropine; biperiden; bromocriptine; cabergoline; citicoline; domperidone; entacapone; levodopa; pimavanserin; piribedil; pramipexole; procyclidine; rasagiline; ropinirole; rotigotine; selegiline; tolcapone; or trihexyphenidyl.

In some embodiments, the additional agent is administered simultaneously with the one or more neurodegenerative therapeutics. In some embodiments, the additional agent is administered sequentially with the one or more neurodegenerative therapeutics. In some embodiments, the additional agent is administered prior to administering the one or more neurodegenerative therapeutics. In some embodiments, the additional agent is administered after administering the one or more neurodegenerative therapeutics.

### Compositions

Compositions of the present invention, including pharmaceutical compositions, include one or more neurodegenerative therapeutics. As used herein, the terms “pharmaceutical composition” and “pharmaceutical formulation” are used interchangeably to refer to a composition suitable for pharmaceutical use in a subject. A pharmaceutical composition generally comprises an effective amount of an active agent, e.g., one or more neurodegenerative therapeutics and a pharmaceutically acceptable carrier, e.g., a buffer, adjuvant, diluent, and the like. In some embodiments, the compositions, including pharmaceutical compositions comprise a concentrated amount of at least one neurodegenerative therapeutic, wherein the concentrated amount is a concentration that is higher than naturally occurring concentrations of the at least one neurodegenerative therapeutic or its naturally occurring counterpart as found in nature.

As used herein, “pharmaceutically acceptable vehicle” and “pharmaceutically acceptable carrier” are used interchangeably and refer to and include any and all solvents, dispersion media, coatings, antibacterial and antifungal agents, isotonic and absorption delaying agents, stabilizers, diluents, suspending agents, thickening agents, excipients, and the like, that are compatible with pharmaceutical administration and comply with applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy. 20^{th} ed. (2000) Lippincott Williams & Wilkins. Baltimore, Md., which is herein incorporated by reference. The pharmaceutically acceptable carrier employed may be either a solid or liquid.

In some embodiments, pharmaceutical compositions disclosed herein comprise a therapeutically effective amount of one or more neurodegenerative therapeutics, and a pharmaceutically acceptable carrier. Pharmaceutical compositions may be prepared in a unit-dosage form appropriate for the desired mode of administration. The pharmaceutical compositions of the present invention may be administered by any suitable route including oral, rectal, nasal, topical (including buccal and sublingual), vaginal, and parenteral (including subcutaneous, intramuscular, intravenous, and intradermal). It will be appreciated that the route of administration may vary with the condition and age of the recipient, the nature of the condition to be treated, and the given compound(s) of the present invention. In some embodiments, the pharmaceutical composition is formulated for oral administration. In some embodiments, the pharmaceutical composition is formulated for parenteral administration. In some embodiments, the pharmaceutical composition is formulated for subcutaneous administration. In some embodiments, the pharmaceutical composition is formulated for intramuscular administration. In some embodiments, the pharmaceutical composition is formulated for intravenous administration. In some embodiments, the pharmaceutical composition is formulated for intraarterial administration. In some embodiments, the pharmaceutical composition is formulated for intradermal administration.

Pharmaceutical compositions are optionally manufactured using methods in the art, such as mixing, dissolving, granulating, dragee-making, levigating, emulsifying, encapsulating, entrapping, or compression processes.

In some embodiments, compositions may also include one or more pH adjusting agents or buffering agents, including acids such as acetic, boric, citric, lactic, phosphoric, and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, pharmaceutical compositions according to the present invention may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate, or bisulfite anions, and include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g., a compound described herein and a co-agent, are both administered to a patient simultaneously in the form of a single entity or dosage. The term “non-fixed combination” means that the active ingredients, e.g., a compound described herein and a co-agent, are administered to a subject as separate entities either simultaneously, concurrently, or sequentially with no specific intervening time limits, wherein such administration provides effective levels of the two compounds in the body of the patient. The latter also applies to cocktail therapy, e.g., the administration of three or more active ingredients.

The pharmaceutical compositions described herein are formulated into any suitable dosage form, including aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the compositions are formulated into capsules. In some embodiments, the compositions are formulated into solutions (for example, for IV administration).

The pharmaceutical solid dosage forms described herein optionally include a compound described herein and one or more pharmaceutically acceptable additives or excipients such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In some embodiments, using standard coating procedures, such as those described in Remington's Pharmaceutical Sciences, 20th Edition (2000), a film coating is provided around the compositions. In some embodiments, the compositions are formulated into particles (for example for administration by capsule) and some or all the particles are coated. In some embodiments, the compositions are formulated into particles (for example for administration by capsule) and some or all the particles are microencapsulated. In some embodiments, the compositions are formulated into particles (for example for administration by capsule) and some or all the particles are not microencapsulated and are uncoated.

In some embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In some embodiments, antioxidants enhance chemical stability where required.

Formulations described herein may benefit from antioxidants, metal chelating agents, thiol containing compounds and other general stabilizing agents. Examples of such stabilizing agents include: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

Examples of a “carrier” or “carrier materials” include excipients in pharmaceutics, which are preferably selected based on compatibility with the given neurodegenerative therapeutic and the release profile properties of the desired dosage form. Exemplary carrier materials include binders, suspending agents, disintegration agents, filling agents, surfactants, solubilizers, stabilizers, lubricants, wetting agents, diluents, and the like. “Pharmaceutically compatible carrier materials” may include acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance), such as phosphate buffered saline solution, are utilized as diluents in the art. In some embodiments, diluents increase bulk of the composition to facilitate compression or create sufficient bulk for homogenous blend for capsule filling. Such compounds include lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a given material in gastrointestinal fluid. Erosion facilitators are generally known in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce, or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per mL, dL, or L of blood serum, absorbed into the bloodstream after administration. As used herein, measurable plasma concentrations are typically measured in ng/ml or μg/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives, and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts, and the like.

### Dosage Forms and Formulations

The pharmaceutical composition of the present invention may be administered by any suitable route including oral, rectal, nasal, topical (including buccal and sublingual), vaginal, and parenteral (including subcutaneous, intramuscular, intravenous, and intradermal). The pharmaceutical composition of the present invention can be formulated into any suitable dosage form, including aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, mixed immediate release and controlled release formulations, and the like.

### Injectable Formulations

The pharmaceutical composition of the present invention can be formulated for intramuscular, subcutaneous, or intravenous injection. Such injectable formulations may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, using a coating such as lecithin, by the maintenance of the required particle size in the case of dispersions, and using surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about using agents delaying absorption, such as aluminum monostearate and gelatin.

For intravenous injections, the one or more neurodegenerative therapeutics may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition may be in a form suitable for parenteral injection as a sterile suspensions, solutions, or emulsions in oily or aqueous vehicles, and may contain formulatory agents such as suspending, stabilizing and/or dispersing agents. Pharmaceutical formulations for parenteral administration include aqueous solutions of the one or more neurodegenerative therapeutics in water-soluble form. Additionally, suspensions of the one or more neurodegenerative therapeutics may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the one or more neurodegenerative therapeutics to allow for the preparation of highly concentrated solutions. Alternatively, the active ingredient may be in powder form for constitution with a suitable vehicle, e.g., sterile pyrogen-free water, before use.

### Oral Formulations

Pharmaceutical preparations for oral use are obtained by mixing one or more solid excipient with one or more neurodegenerative therapeutics, optionally grinding the resulting mixture, and processing the mixture of granules, after adding suitable auxiliaries, if desired, to obtain tablets or dragee cores. Suitable excipients include filling agents such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate.

Dragee cores can be provided with suitable coatings. For this purpose, concentrated sugar solutions can be used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Solid dosage forms can be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In some embodiments, a pharmaceutical composition of the present invention is in the form of a powder. In some embodiments, a pharmaceutical composition of the present invention is in the form of a tablet, including a fast-melt tablet.

In some embodiments, a pharmaceutical composition of the present invention is in a solid dosage form and includes a pharmaceutically acceptable carrier, binder, filler suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or a combination of one or more thereof. Suitable carriers for use in the solid dosage forms include acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol, and the like. Suitable filling agents for use in the solid dosage forms include lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like. Suitable binders include carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like. Suitable lubricants or glidants include stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like. Suitable diluents include sugars (including lactose, sucrose, and dextrose), polysaccharides (including dextrates and maltodextrin), polyols (including mannitol, xylitol, and sorbitol), cyclodextrins and the like. Suitable wetting agents include oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS, and the like. Suitable surfactants include sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Suitable suspending agents include polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone, and the like. Suitable antioxidants include butylated hydroxytoluene (BHT), sodium ascorbate, and tocopherol.

In some embodiments, a pharmaceutical composition of the present invention is provided as a liquid formulation for oral administration, which includes oral dispersions, emulsions, solutions, elixirs, gels, and syrups. See, e.g., Singh et al., Encyclopedia of Pharmaceutical Technology, 2nd Ed., pp. 754-757 (2002). In some embodiments, the liquid formulations include additives, such as: (a) disintegrating agents; (b) dispersing agents; (c) wetting agents; (d) preservatives, (e) viscosity enhancing agents, (f) sweetening agents, and (g) flavoring agents. In some embodiments, the liquid formulations further include a crystalline inhibitor. In some embodiments, the liquid formulations remain in a homogenous state, as defined in The USP Pharmacists' Pharmacopeia (2005 edition, chapter 905), for at least 4 hours. The homogeneity can be determined by a sampling method consistent with determining homogeneity of the entire composition. In some embodiments, the liquid formulation is re-suspended into a homogenous state by physical agitation lasting less than 1 minute. In some embodiments, the liquid formulation is re-suspended into a homogenous state by physical agitation lasting less than 45 seconds. In some embodiments, the liquid formulation is re-suspended into a homogenous state by physical agitation lasting less than 30 seconds. In some embodiments, no agitation is necessary to maintain a homogeneous state.

In some embodiments, a pharmaceutical composition of the present invention is in a microencapsulated formulation. In some embodiments, the microencapsulated formulations include one or more materials selected from pH modifiers, erosion facilitators, anti-foaming agents, antioxidants, flavoring agents, and pharmaceutically acceptable carriers. In some embodiments, the microencapsulated material delays the release of the one or more neurodegenerative therapeutics. Exemplary microencapsulation materials useful for delaying the release of the one or more neurodegenerative therapeutics include hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and combinations of one or more thereof. Microencapsulated formulations according to the present invention may be made using methods known in the art.

### Dosing and Treatment Regimens

In some embodiments, the amount of the one or more neurodegenerative therapeutics administered to the subject is a therapeutically effective amount. A “therapeutically effective amount”, refers to an amount of one or more neurodegenerative therapeutics that, when administered to a subject (i) treats or inhibits the given neurodegenerative disease or disorder, (ii) attenuates, ameliorates, or eliminates one or more symptoms of the given neurodegenerative disease or disorder, and/or (iii) inhibits or delays the onset of one or more symptoms of the neurodegenerative disease or disorder, as compared to a control. A therapeutically effective amount of one or more neurodegenerative therapeutic will vary depending upon factors such as the given compound(s), the pharmaceutical formulation, route of administration, the specific neurodegenerative disease or disorder, the degree of severity of the neurodegenerative disease or disorder, and the identity of the subject being treated, but can nevertheless be readily determined by one skilled in the art. For example, a “therapeutically effective amount” of a neurodegenerative therapeutic is one that inhibits or reduces a symptom of an neurodegenerative disease or disorder as compared to a negative control.

In some embodiments, one or more neurodegenerative therapeutics are administered prior to, during, and/or after the onset of one or more symptoms of a neurodegenerative disease or disorder. In some embodiments, a therapeutically effective amount one or more neurodegenerative therapeutics is administered prior to, during, and/or after the onset of one or more symptoms of a neurodegenerative disease or disorder.

The one or more neurodegenerative therapeutics to be administered to a subject may be provided in the form of a pharmaceutical composition. Preferred pharmaceutical compositions are those comprising at least one neurodegenerative therapeutic in a therapeutically effective amount, and a pharmaceutically acceptable vehicle. Pharmaceutical compositions may be prepared in a unit-dosage form appropriate for the desired mode of administration.

The actual dosages of the one or more neurodegenerative therapeutics will vary according to the specific compound(s) being used, the given pharmaceutical formulation, the mode of administration, and the given treatment site, subject, and disease being treated. Optimal dosages for a given set of conditions may be ascertained by those skilled in the art using dosage determination tests in view of the experimental data for a given compound. Administration of prodrugs may be dosed at weight levels that are chemically equivalent to the weight levels of the fully active forms.

In some embodiments, a therapeutically effective amount of one or more neurodegenerative therapeutics is administered as a daily dose of about 0.01-2, about 0.25-2, about 0.5-2, about 1-2, or about 2 grams per kilogram weight of the subject per day. In some embodiments, a therapeutically effective amount of one or more neurodegenerative therapeutics is administered as a daily dose of about 0.1-1, about 0.25-1, about 0.5-1, or about 1 gram per kilogram weight of the subject per day. In some embodiments, a therapeutically effective amount of one or more neurodegenerative therapeutics is administered as a daily dose of about 0.01-1.0, about 0.01-0.5, or about 0.1-0.2 grams per kilogram weight of the subject per day. The skilled artisan will appreciate that certain factors may influence the dosage required to effectively treat a subject, including the severity of the neurodegenerative disease or disorder, previous treatments, the general health and/or age of the subject, and other diseases present.

In some embodiments, the therapeutically effective amount may be administered as a single dose or as multiple doses (e.g., 2, 3, 4, 5, 6, 7, 8, 9, 10, or more doses) over a given period. For example, a subject may be treated with one or more neurodegenerative therapeutics at least once. Alternatively, the subject may be treated with one or more neurodegenerative therapeutics from about one time per week to about once daily for a given treatment period. The length of the treatment period will depend on a variety of factors such as the severity of the neurodegenerative disease or disorder and/or the concentration and activity of the one or more neurodegenerative therapeutics. It will also be appreciated that the effective dosage of the one or more neurodegenerative therapeutics may increase or decrease over the course of a given treatment.

Toxicity and therapeutic efficacy of the one or more neurodegenerative therapeutics can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds exhibiting large therapeutic indices are preferred. While compounds that exhibit toxic side effects may be used, care should be taken to design a delivery system that targets such compounds to the site of affected tissue to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### Kits and Articles of Manufacture

In some embodiments, the present invention provides kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some embodiments, the containers are formed from a variety of materials such as glass or plastic. In some embodiments, articles of manufacture according to the present invention contain packaging materials. Examples of packaging materials include blister packs, bottles, tubes, bags, containers, bottles, and packaging materials suitable for the given pharmaceutical formulation and intended mode of administration and treatment.

In some embodiments, the present invention is directed to kits which comprise one or more neurodegenerative therapeutics, optionally in a composition or in combination with one or more additional therapeutic agents, packaged together with one or more reagents or drug delivery devices for preventing, inhibiting, reducing, or treating an neurodegenerative disease or disorder. Such kits include a carrier, package, or container that may be compartmentalized to receive one or more containers, such as vials, tubes, and the like. In some embodiments, the kits optionally include an identifying description or label and/or instructions relating to its use. In some embodiments, the kits comprise the one or more neurodegenerative therapeutics, optionally in one or more unit dosage forms, packaged together as a pack, e.g., a blister pack, and/or in drug delivery device, e.g., a pre-filled syringe. In some embodiments, the kits include information prescribed by a governmental agency that regulates the manufacture, use, or sale of compounds and compositions according to the present invention.

### EXAMPLES

The following examples are intended to illustrate but not to limit the invention.

Materials: α KG (75890, Sigma), 2-HB (220116, Sigma), 3-HB (54920, Sigma), α KB (K401, Sigma), KIC (K0629, Sigma), L-alanine (A4349, Sigma), KIV (198994, Sigma), Pyruvate (107360, Sigma).

### Example 1

To investigate the effects of small molecules in a C. elegans model of Alzheimer's disease, the GMC101 C. elegans strain, developed by Gawain McColl at the University of Melbourne, Australia, was used. The GMC101 C. elegans strain expresses the full length human amyloid beta 1-42 protein in the body wall muscle cells, leading to a fully penetrant age-progressive paralysis. These worms were first age-synchronized by performing an egg prep (mixed <100 gravid worms in 70 μl M9 buffer, 25 μl bleach and 5 μl 10 N NaOH). Once the worms reached day 1 of adulthood, they were picked onto NGM treatment plates containing 49.5 μM 5-fluoro-2′-deoxyuridine (FUDR) to prevent progeny production and either compounds or vehicle (water) control. The worms were then shifted to 25° C. to induce amyloid-beta aggregation and assessed for paralysis daily by the failure to perform whole body bends or significantly move forwards and backwards upon gentle prodding with a platinum wire. All worms were transferred to fresh plates on Day 5. 8 mM α-KG was observed to delay paralysis by 22% compared to vehicle treatment.

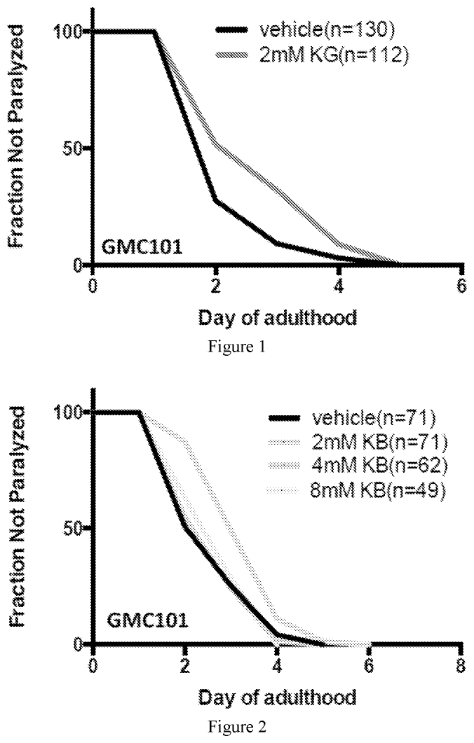

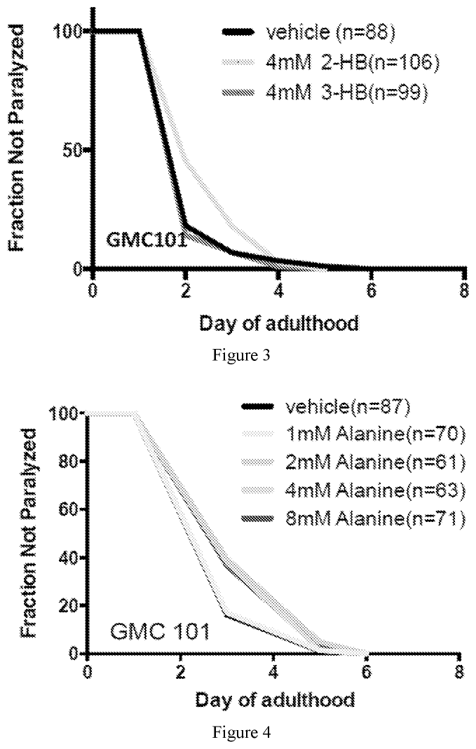

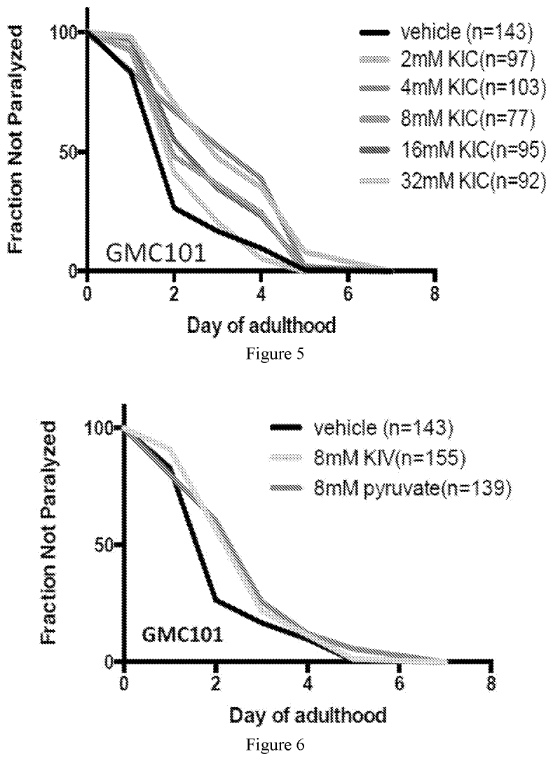

FIG. 1-FIG. 6 illustrate a decrease in Aβ toxicity and delayed paralysis in GMC101 C. elegans strain induced by small molecules. FIG. 1 shows anti-aging compound, α-ketoglutarate (α-KG) can extend the health span of GMC101 C. elegans strain at 2 mM. FIG. 2 shows α-ketobutyrate (α-KB) can delay Aβ caused paralysis, e.g., at a concentration of about 2 mM. FIG. 3 shows an exemplary α-KB analog, 2-hydroxybutyrate (2-HB) can also delay GMC101 paralysis at 4 mM. A delay in paralysis is not observed with 3-hydroxybutyrate (3-HB). FIG. 4 shows that alanine can reduce paralysis caused by Aβ. FIG. 5 shows that the metabolite of leucine, α-ketoisocaproate (KIC) reduces paralysis caused by Aβ. FIG. 6 shows that pyruvate reduces Aβ toxicity and delays paralysis. The metabolite of valine, α-ketoisovalerate (KIV) can extend GMC101 health span.

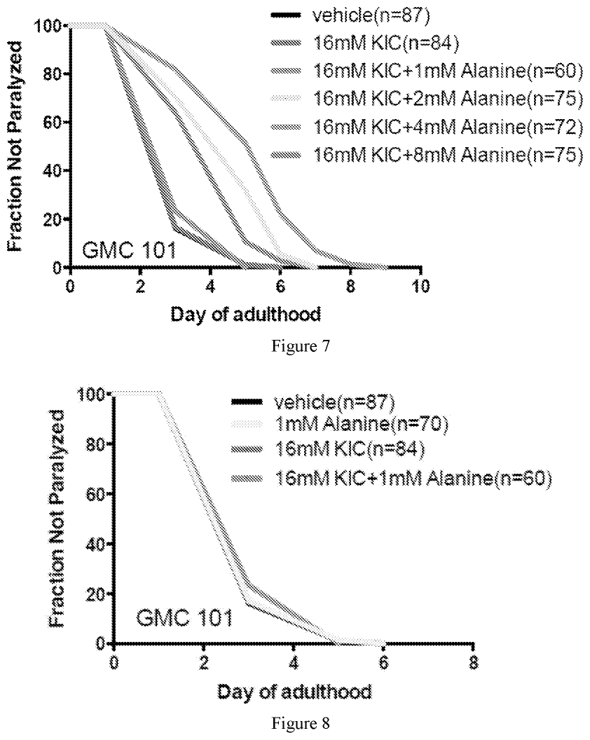

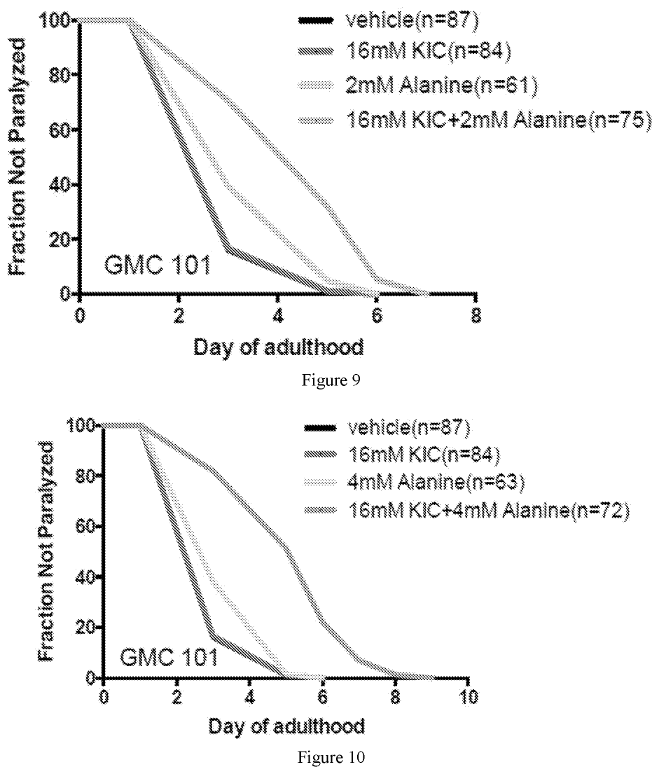

FIG. 7-FIG. 11 illustrate that 16 mM KIC can synergize with alanine to decrease Aβ toxicity and delay paralysis in GMC101 C. elegans strain. FIG. 7 shows that 16 mM KIC can delay paralysis when treated together with alanine, while 16 mM KIC is not observed to exhibit beneficial effects in GMC101. FIG. 8 shows that 1 mM alanine does not synergize with 16 mM KIC in GMC101. FIG. 9, FIG. 10, and FIG. 11 show 2 mM, 4 mM, or 8 mM alanine can extend health span of GMC101 and synergize with 16 mM KIC to reduce paralysis caused by Aβ.

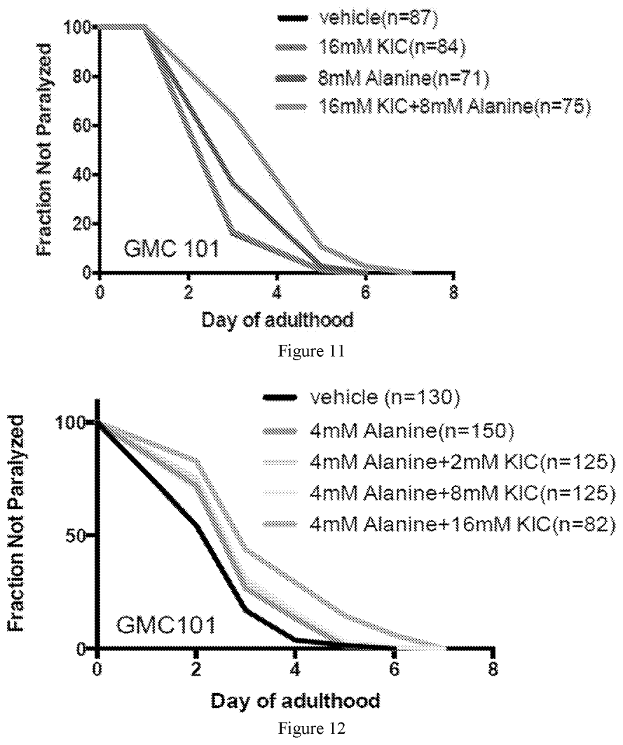

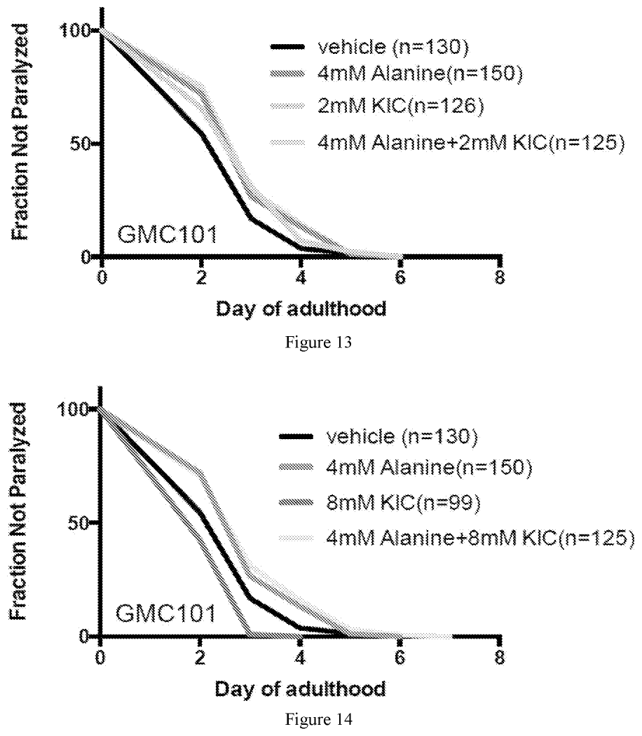

FIG. 12-FIG. 15 show 4 mM alanine can synergize with KIC only at high concentration to decrease Aβ toxicity and delay paralysis in GMC101 C. elegans strain. FIG. 12 shows 4 mM alanine can delay paralysis when treated together with 16 mM KIC, while lower concentrations of KIC is not observed to exert a beneficial effect in combination with alanine. FIG. 13 and FIG. 14 show 2 mM or 8 mM KIC does not synergize with 4 mM alanine in GMC101. FIG. 15 shows 16 mM KIC does not exert a beneficial effect in GMC101 but is observed to synergize with 4 mM alanine to reduce paralysis caused by Aβ.

### Example 2

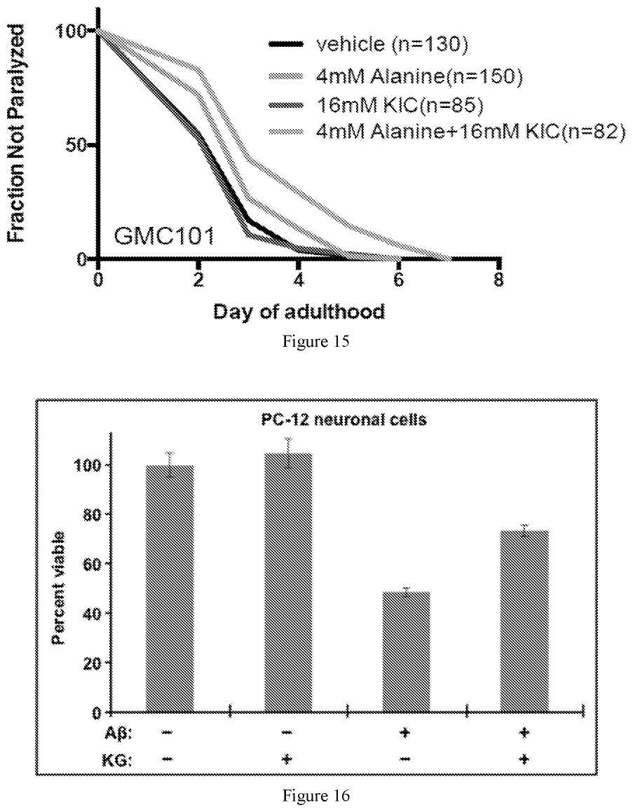

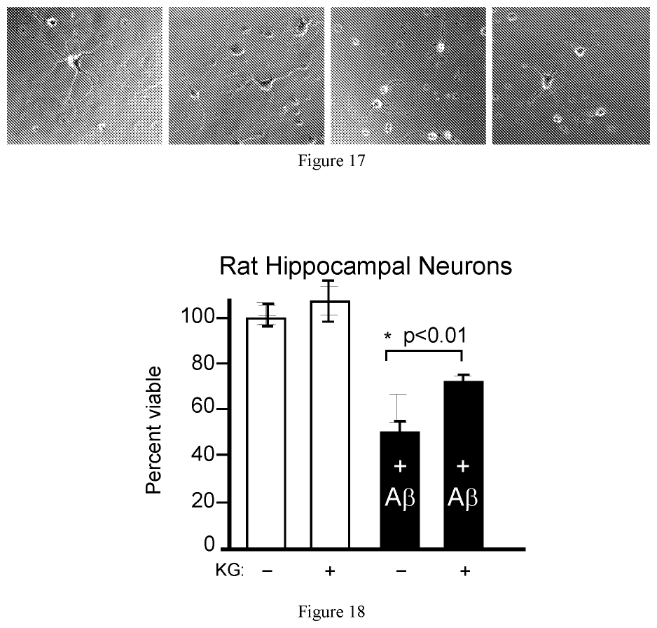

To show that α-KG prevents amyloid-beta induced cell death in neuronal cell culture, PC-12 cells were seeded on poly-Lys-coated 96 well plates at 2,000 cells/well in 10% FCS in RPMI 1640 culture medium supplemented with Penicillin/Streptomycin antibiotics. Overnight after seeding, cells were differentiated with NGF in 0.5% FCS, RPMI 1640 medium for 7 days. Cells were pretreated with 200 μM octyl α-KG for 2 hours, before Aβ42 or Aβ25-35 were added. FIG. 16 shows that after a 24-hour incubation, only about 48.5±1.8% of control cells were viable, whereas about 73±2.2% of octyl α-KG treated cells remained alive. These results indicate that α-KG protects differentiated PC-12 cells against Aβ induced cell death.

Comparable results were obtained using primary neurons. Primary rat and mouse hippocampal neurons were isolated from the hippocampi of 1-2 day postnatal pups or 8-week old adult mice. Hippocampi were rinsed and minced to small pieces in HBSS buffer containing Penicillin/Streptomycin antibiotics and 10 mM HEPES pH 7.3, digested in 0.05% Trypsin at 37° C. for 15 minutes and in DNase I for 5 minutes, washed twice with HBSS buffer, and neurons dissociated by trituration with a fire-polished glass pipette. The hippocampal neuronal cells were cultured in Neurobasal A medium supplemented with B27 on poly-D-Lys coated plates. The medium was changed twice a week. After 7 days in culture, cells were pretreated with octyl α-KG at dose of 50-500 μM for 2 hours. Aβ15-25 peptide was added into the culture for 24-48 hours to induce apoptosis and neuronal death. Cell morphology was examined and cell viability was assessed using Alamar Blue assay. FIG. 17 shows that octyl α-KG protects primary mouse hippocampal neurons against Aβ induced neuronal dysfunction and cell death. FIG. 18 shows that octyl α-KG protects primary rat hippocampal neurons against Aβ induced cell death.

### Additional Embodiments

### Embodiment 1

A method of treating a neurodegenerative disease or disorder in a subject in need thereof, which comprises

administering to the subject a pharmaceutical composition comprising:

a therapeutically effective amount of a compound of Formula I:

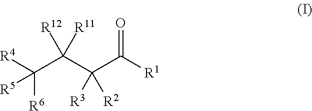

wherein:

R¹ is hydrogen, halogen, —CHO, —OR⁷, —NR⁸R⁹, —COOR⁷, —CONR⁸R⁹, —SR¹⁰, substituted or unsubstituted alkyl, or substituted or unsubstituted heteroalkyl;

R² and R³ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl, or R² and R³, together with the atom to which they are bound, form an oxo;

R¹¹ and R¹² are each independently hydrogen, halogen, —CN, —CHO, —OR¹³, —NR¹⁴R¹⁵, —COOR¹³, —CONR¹⁴R¹⁵, —NO₂, —SR¹⁶, substituted or unsubstituted alkyl, or substituted or unsubstituted heteroalkyl;

R⁴, R⁵, and R⁶ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl;

R⁷, R⁸, R⁹, and R¹⁰ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

R¹³, R¹⁴, R¹⁵ and R¹⁶ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or a salt thereof.

### Embodiment 2

The method of Embodiment 1, wherein R¹ is hydrogen, —CHO, or —OR⁷.

### Embodiment 3

The method of Embodiment 2, wherein R¹ is —OR⁷, wherein R⁷ is hydrogen, substituted or unsubstituted alkyl, or substituted or unsubstituted aryl.

### Embodiment 4

The method of Embodiment 3, wherein R⁷ is hydrogen.

### Embodiment 5

The method of Embodiment 3, wherein R¹ is —OR⁷, wherein R⁷ is C₁₋₂₀ substituted or unsubstituted alkyl.

### Embodiment 6

The method of Embodiment 1, wherein R² is hydrogen, halogen, —CN, —CHO, —OR⁷, or —NR⁸R⁹, wherein R⁷, R⁸ and R⁹ are each independently hydrogen or substituted or unsubstituted alkyl.

### Embodiment 7

The method of Embodiment 6, wherein R² is hydrogen, —OR⁷, or —NR⁸R⁹, wherein R⁷, R⁸ and R⁹ are each independently hydrogen or substituted or unsubstituted alkyl.

### Embodiment 8

The method of Embodiment 1, wherein R³ is hydrogen, halogen, —CN, —CHO, —OR⁷, or —NR⁸R⁹, wherein R⁷, R⁸ and R⁹ are each independently hydrogen or substituted or unsubstituted alkyl.

### Embodiment 9

The method of Embodiment 8, wherein R³ is hydrogen, —OR⁷, or —NR⁸R⁹, wherein R⁷, R⁸ and R⁹ are each independently hydrogen or substituted or unsubstituted alkyl.

### Embodiment 10

The method of Embodiment 1, wherein R² and R³, together with the atom to which they are bound, form an oxo.

### Embodiment 11

The method of Embodiment 1, wherein R¹¹ is hydrogen, halogen, —CN, —CHO, —OR¹³, —NR¹⁴R¹⁵, or substituted or unsubstituted alkyl, wherein R¹³, R¹⁴ and R¹⁵ are each independently hydrogen or substituted or unsubstituted alkyl.

### Embodiment 12

The method of Embodiment 11, wherein R¹¹ is hydrogen, —OR¹³, or —NR¹⁴R¹⁵, wherein R¹³, R¹⁴ and R¹⁵ are each independently hydrogen or substituted or unsubstituted alkyl.

### Embodiment 13

The method of Embodiment 1, wherein R¹² is hydrogen, halogen, —CN, —CHO, —OR¹³, —NR¹⁴R¹⁵, or substituted or unsubstituted alkyl, wherein R¹³, R¹⁴ and R¹⁵ are each independently hydrogen or substituted or unsubstituted alkyl.

### Embodiment 14

The method of Embodiment 13, wherein R¹² is hydrogen, —OR¹³, or —NR¹⁴R¹⁵, wherein R¹³, R¹⁴ and R¹⁵ are each independently hydrogen or substituted or unsubstituted alkyl.

### Embodiment 15

The method of Embodiment 1, wherein R⁴, R⁵, and R⁶ are each independently hydrogen, —CHO, —OR⁷, —NR⁸R⁹, —COOR⁷, or —CONR⁸R⁹, wherein R⁷, R⁸, and R⁹ are each independently hydrogen or C₁₋₂₀ substituted or unsubstituted alkyl.

### Embodiment 16

The method of Embodiment 15, wherein R⁴ is —COOR⁷ or —CONR⁸R⁹, wherein R⁷, R⁸, and R⁹ are each independently hydrogen or C₁₋₂₀ substituted or unsubstituted alkyl.

### Embodiment 17

The method of Embodiment 15, wherein R⁵ and R⁶ are each independently hydrogen, —OR⁷, or —CONR⁸R⁹, wherein R⁷, R⁸, and R⁹ are each independently hydrogen or C₁₋₂₀ substituted or unsubstituted alkyl.

### Embodiment 18

The method of Embodiment 1, wherein the neurodegenerative disease or disorder is a neurodegenerative disease of the central nervous system.

### Embodiment 19

The method of Embodiment 1 or 18, wherein the neurodegenerative disease or disorder is Alzheimer's disease, Parkinson's disease, or Huntington disease.

### Embodiment 20

The method of Embodiment 1 or 18, wherein the neurodegenerative disease or disorder is Alzheimer's disease.

### Embodiment 21

The method of any one of the Embodiments 1-17, wherein the compound of Formula I is alpha-ketoglutarate (α KG).

### Embodiment 22

The method of any one of the Embodiments 1-17, wherein the compound of Formula I is 2-hydroxybutyrate (2-HB).

### Embodiment 23

The method of any one of the Embodiments 1-17, wherein the compound of Formula I is alpha-ketobutyrate (α KB).

### Embodiment 24

The method of any one of the Embodiments 1-17, wherein the compound of Formula I is α ketoisocaproate (KIC).

### Embodiment 25

The method of any one of the Embodiments 1-17, wherein the compound of Formula I is α ketoisovalerate (KIV).

### Embodiment 26

The method of Embodiment 21, wherein the concentration of a KG is about 1 μM to about 16 mM.

### Embodiment 27

The method of Embodiment 21 or 26, wherein the concentration of a KG is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, or about 16 mM.

### Embodiment 28

The method of Embodiment 22, wherein the concentration of 2-HB is about 1 μM to about 16 mM.

### Embodiment 29

The method of Embodiment 22 or 28, wherein the concentration of 2-HB is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, or about 16 mM.

### Embodiment 30

The method of Embodiment 23, wherein the concentration of α KB is about 1 μM to about 16 mM.

### Embodiment 31

The method of Embodiment 23 or 30, wherein the concentration of α KB is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, or about 16 mM.

### Embodiment 32

The method of Embodiment 24, wherein the concentration of KIC is about 1 μM to about 32 mM.

### Embodiment 33

The method of Embodiment 24 or 32, wherein the concentration of KIC is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, about 15 mM, about 16 mM, about 20 mM, about 24 mM, about 25 mM, about 28 mM, about 30 mM, or about 32 mM.

### Embodiment 34

The method of Embodiment 25, wherein the concentration of KIV is about 1 μM to about 32 mM.

### Embodiment 35

The method of Embodiment 25 or 34, wherein the concentration of KIV is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, about 15 mM, about 16 mM, about 20 mM, about 24 mM, about 25 mM, about 28 mM, about 30 mM, or about 32 mM.

### Embodiment 36

The method of any one of the Embodiments 1-35, further comprising administering to the subject a pharmaceutical composition comprising alanine.

### Embodiment 37

The method of Embodiment 36, wherein the concentration of alanine is about 1 μM to about 16 mM.

### Embodiment 38

The method of any one of the Embodiments 1 to 37, wherein KIC is administered simultaneously with alanine.

### Embodiment 39

The method of any one of the Embodiments 1 to 37, wherein KIC is administered sequentially with alanine.

### Embodiment 40

The method of any one of the Embodiments 1 to 37, wherein KIV is administered simultaneously with alanine.

### Embodiment 41

The method of any one of the Embodiments 1 to 37, wherein KIV is administered sequentially with alanine.

### Embodiment 42

The method of any one of the Embodiments 1 to 41, wherein the compound of Formula I is formulated for oral or parenteral administration.

### Embodiment 43

The method of any one of the Embodiments 1 to 42, wherein the therapeutically effective amount is administered as a single dose.

### Embodiment 44

The method of any one of the Embodiments 1 to 42, wherein the therapeutically effective amount is administered in at least two doses, at least three doses, at least four doses, at least five doses, or more.

### Embodiment 45

The method of any one of the Embodiments 1 to 44, wherein the therapeutically effective amount is administered daily.

### Embodiment 46

The method of any one of the Embodiments 1 to 44, wherein the therapeutically effective amount is administered every other day.

### Embodiment 47

The method of any one of the Embodiments 1 to 46, wherein the subject is a human.

### Embodiment 48

A method of treating a neurodegenerative disease or disorder in a subject in need thereof, comprising: administering to the subject a first pharmaceutical composition comprising a ketoisocaproate (KIC) or a ketoisovalerate (KIV), and an excipient.

### Embodiment 49

The method of Embodiment 48, further comprising administering to the subject a second pharmaceutical composition comprising alanine.

### Embodiment 50

The method of Embodiment 48, wherein the concentration of KIC is about 1 μM to about 32 mM.

### Embodiment 51

The method of Embodiment 48 or 50, wherein the concentration of KIC is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, about 15 mM, about 16 mM, about 20 mM, about 24 mM, about 25 mM, about 28 mM, about 30 mM, or about 32 mM.

### Embodiment 52

The method of Embodiment 48, wherein the concentration of KIV is about 1 μM to about 32 mM.

### Embodiment 53

The method of Embodiment 48 or 52, wherein the concentration of KIV is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, about 15 mM, about 16 mM, about 20 mM, about 24 mM, about 25 mM, about 28 mM, about 30 mM, or about 32 mM.

### Embodiment 54

The method of Embodiment 49, wherein the concentration of alanine is about 1 μM to about 16 mM.

### Embodiment 55

The method of any one of the Embodiments 48 to 54, wherein

KIC is administered simultaneously with alanine.

### Embodiment 56

The method of any one of the Embodiments 48 to 54, wherein

KIC is administered sequentially with alanine.

### Embodiment 57

The method of any one of the Embodiments 48 to 54, wherein

KIV is administered simultaneously with alanine.

### Embodiment 58

The method of any one of the Embodiments 48 to 54, wherein

KIV is administered sequentially with alanine.

### Embodiment 59

The method of Embodiment 48, wherein the neurodegenerative disease or disorder is a neurodegenerative disease of the central nervous system.

### Embodiment 60

The method of Embodiment 48 or 59, wherein the neurodegenerative disease or disorder is Alzheimer's disease, Parkinson's disease, or Huntington disease.

### Embodiment 61

The method of Embodiment 48 or 59, wherein the neurodegenerative disease or disorder is Alzheimer's disease.

### Embodiment 62

The method of any one of the Embodiments 48 to 61, wherein the first pharmaceutical composition is formulated for oral or parenteral administration.

### Embodiment 63

The method of any one of the Embodiments 48 to 62, wherein the second pharmaceutical composition is formulated for oral or parenteral administration.

### Embodiment 64

The method of any one of the Embodiments 48 to 63, wherein the first pharmaceutical composition and/or the second pharmaceutical composition is administered as a therapeutically effective amount.

### Embodiment 65

The method of any one of the Embodiments 48 to 64, wherein the therapeutically effective amount is administered as a single dose.

### Embodiment 66

The method of any one of the Embodiments 48 to 64, wherein the therapeutically effective amount is administered in at least two doses, at least three doses, at least four doses, at least five doses, or more.

### Embodiment 67

The method of any one of the Embodiments 48 to 66, wherein the therapeutically effective amount is administered daily.

### Embodiment 68

The method of any one of the Embodiments 48 to 66, wherein the therapeutically effective amount is administered every other day.

### Embodiment 69

The method of any one of the Embodiments 48 to 68, wherein the subject is a human.

All scientific and technical terms used in this application have meanings commonly used in the art unless otherwise specified.

The section headings used herein are for organizational purposes and are not to be construed as limiting the subject matter described.

As used herein, the terms “subject”, “patient”, and “individual” are used interchangeably to refer to humans and non-human animals. The term “non-human animal” includes all vertebrates, e.g., mammals and non-mammals, such as non-human primates, horses, sheep, dogs, cows, pigs, chickens, and other veterinary subjects and test animals. In some embodiments, the subject is a mammal. In some embodiments, the subject is a human.

The use of the singular can include the plural unless specifically stated otherwise. As used in the specification and the appended claims, the singular forms “a”, “an”, and “the” can include plural referents unless the context clearly dictates otherwise. The use of “or” can mean “and/or” unless stated otherwise. As used herein, “and/or” means “and” or “or”. For example, “A and/or B” means “A, B, or both A and B” and “A, B, C, and/or D” means “A, B, C, D, or a combination thereof” and said “combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method of treating a neurodegenerative disease or disorder in a subject in need thereof, which comprises
administering to the subject a pharmaceutical composition comprising:
a therapeutically effective amount of a compound of Formula I: 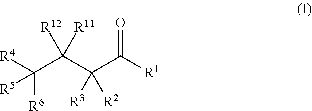
wherein:
R¹ is hydrogen, halogen, —CHO, —OR⁷, —NR⁸R⁹, —COOR⁷, —CONR⁸R⁹, —SR¹⁰, substituted or unsubstituted alkyl, or substituted or unsubstituted heteroalkyl;
R² and R³ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl, or R² and R³, together with the atom to which they are bound, form an oxo;
R¹¹ and R¹² are each independently hydrogen, halogen, —CN, —CHO, —OR¹³, —NR¹⁴R¹⁵, —COOR¹³, —CONR¹⁴R¹⁵, —NO₂, —SR¹⁶, substituted or unsubstituted alkyl, or substituted or unsubstituted heteroalkyl;
R⁴, R⁵, and R⁶ are each independently hydrogen, halogen, —CN, —CHO, —OR⁷, —NR⁸R⁹, —COOR⁷, —CONR⁸R⁹, —NO₂, —SR¹⁰, substituted or unsubstituted alkyl, or substituted or unsubstituted heteroalkyl;
R⁷, R⁸, R⁹, and R¹⁰ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R¹³, R¹⁴, R¹⁵, and R¹⁶ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or a salt thereof.

2. The method of claim 1, wherein R¹ is hydrogen, —CHO, or —OR⁷; R¹ is —OR⁷, and R⁷ is hydrogen, substituted or unsubstituted alkyl, or substituted or unsubstituted aryl; R¹ is —OR⁷, and R⁷ is hydrogen; or R¹ is —OR⁷, and R⁷ is C₁₋₂₀ substituted or unsubstituted alkyl.

3. The method of claim 1, wherein R² is hydrogen, halogen, —CN, —CHO, —OR⁷, or —NR⁸R⁹, and R⁷, R⁸ and R⁹ are each independently hydrogen or substituted or unsubstituted alkyl; or R² is hydrogen, —OR⁷, or —NR⁸R⁹, and R⁷, R⁸ and R⁹ are each independently hydrogen or substituted or unsubstituted alkyl.

4. The method of claim 1, wherein R³ is hydrogen, halogen, —CN, —CHO, —OR⁷, or —NR⁸R⁹, and R⁷, R⁸ and R⁹ are each independently hydrogen or substituted or unsubstituted alkyl; or R³ is hydrogen, —OR⁷, or —NR⁸R⁹, and R⁷, R⁸ and R⁹ are each independently hydrogen or substituted or unsubstituted alkyl.

5. The method of claim 1, wherein R² and R³, together with the atom to which they are bound, form an oxo.

6. The method of claim 1, wherein R¹¹ is hydrogen, halogen, —CN, —CHO, —OR¹³, —NR¹⁴R¹⁵, or substituted or unsubstituted alkyl, wherein R¹³, R¹⁴ and R¹⁵ are each independently hydrogen or substituted or unsubstituted alkyl; preferably R¹¹ is hydrogen, —OR¹³, or —NR¹⁴R¹⁵, wherein R¹³, R¹⁴ and R¹⁵ are each independently hydrogen or substituted or unsubstituted alkyl.

7. The method of claim 1, wherein R¹² is hydrogen, halogen, —CN, —CHO, —OR¹³, —NR¹⁴R¹⁵, or substituted or unsubstituted alkyl, wherein R¹³, R¹⁴ and R¹⁵ are each independently hydrogen or substituted or unsubstituted alkyl; preferably R¹² is hydrogen, —OR¹³, or —NR¹⁴R¹⁵, wherein R¹³, R¹⁴ and R¹⁵ are each independently hydrogen or substituted or unsubstituted alkyl.

8. The method of claim 1, wherein R⁴, R⁵, and R⁶ are each independently hydrogen, —CHO, —OR⁷, —NR⁸R⁹, —COOR⁷, or —CONR⁸R⁹, wherein R⁷, R⁸, and R⁹ are each independently hydrogen or C₁₋₂₀ substituted or unsubstituted alkyl;
R⁴ is —COOR⁷ or —CONR⁸R⁹, and R⁵ and R⁶ are each independently hydrogen, —CHO, —OR⁷, —NR⁸R⁹, —COOR⁷, or —CONR⁸R⁹, wherein R⁷, R⁸, and R⁹ are each independently hydrogen or C₁₋₂₀ substituted or unsubstituted alkyl; or
R⁴ is hydrogen, —CHO, —OR⁷, —NR⁸R⁹, —COOR⁷, or —CONR⁸R⁹, and R⁵ and R⁶ are each independently hydrogen, —OR⁷, or —CONR⁸R⁹, wherein R⁷, R⁸, and R⁹ are each independently hydrogen or C₁₋₂₀ substituted or unsubstituted alkyl.

9. The method of claim 1, wherein the neurodegenerative disease or disorder is a neurodegenerative disease of the central nervous system, such as Alzheimer's disease, Parkinson's disease, or Huntington disease.

10. The method of claim 1, wherein the neurodegenerative disease or disorder is Alzheimer's disease.

11. The method of claim 1, wherein the compound of Formula I is alpha-ketoglutarate (α-KG), 2-hydroxybutyrate (2-HB), alpha-ketobutyrate (α-KB), α-ketoisocaproate (KIC), or α-ketoisovalerate (KIV).

12. The method of claim 1, wherein the compound of Formula I is α-ketoisocaproate (KIC) or α-ketoisovalerate (KIV) and the pharmaceutical composition further comprises an excipient.

13. The method of claim 11, wherein
the concentration of α-KG is about 1 μM to about 16 mM;
the concentration of α-KG is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, or about 16 mM;
the concentration of 2-HB is about 1 μM to about 16 mM;
the concentration of 2-HB is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, or about 16 mM;
the concentration of α-KB is about 1 μM to about 16 mM;
the concentration of α-KB is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, or about 16 mM;
the concentration of KIC is about 1 μM to about 32 mM;
the concentration of KIC is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, about 15 mM, about 16 mM, about 20 mM, about 24 mM, about 25 mM, about 28 mM, about 30 mM, or about 32 mM;
the concentration of KIV is about 1 μM to about 32 mM; or
the concentration of KIV is about 1 μM, about 10 μM, about 100 μM, about 500 μM, about 1 mM, about 1.5 mM, about 2 mM, about 2.5 mM, about 3 mM, about 4 mM, about 5 mM, about 8 mM, about 10 mM, about 15 mM, about 16 mM, about 20 mM, about 24 mM, about 25 mM, about 28 mM, about 30 mM, or about 32 mM.

14. The method claim 1, further comprising administering to the subject a pharmaceutical composition comprising alanine.

15. The method of claim 1, wherein the concentration of alanine is about 1 μM to about 16 mM.

16. The method of claim 1, wherein KIC is administered simultaneously or sequentially with alanine; or KIV is administered simultaneously or sequentially with alanine.

17. The method of claim 1, wherein the pharmaceutical composition comprising the compound of Formula I is formulated for oral or parenteral administration and/or the pharmaceutical composition comprising alanine is formulated for oral or parenteral administration.

18. The method of claim 1, wherein the pharmaceutical composition comprising the compound of Formula I and/or the pharmaceutical composition comprising alanine is administered as a therapeutically effective amount.

19. The method of claim 1, wherein the therapeutically effective amount is administered as a single dose or as multiple doses, for example, at least two doses, at least three doses, at least four doses, at least five doses, or more.

20. The method of claim 1, wherein the therapeutically effective amount is administered daily or every other day.

21. The method of claim 1, wherein the subject is a human.
